(19) United States
(12) Patent Application Publication
(10) Pub. No.: US 2002/0187508 A1
(43) Pub. Date: Dec. 12, 2002

(54) Methods and products for analyzing nucleic acids using nick translation

(76) Inventor: Gordon G. Wong, Brookline, MA (US)

Correspondence Address: Maria A. Trevisan, Wolf, Greenfield & Sacks, P.C., Federal Reserve Plaza, 600 Atlantic Avenue, Boston, MA, 02210, US

(21) Appl. No.: 10/166,567

(22) Filed: Jun. 10, 2002

Related U.S. Application Data
(60) Provisional application No. 60/297,080, filed on Jun. 8, 2001.

(51) Int. Cl. C12Q 1/68; C12P 19/34

(52) U.S. Cl. 435/6; 435/91.2

(57) ABSTRACT

The invention relates to methods, products and systems for analyzing nucleic acid molecules using sequence specific nick translation. The methods can be used to obtain sequence information about the nucleic acid molecules and to assess the efficacy of therapeutic treatments that affect based on DNA damage induction.

### RELATED APPLICATIONS

This application claims priority to U.S. Provisional Patent Application filed Jun. 8, 2001, entitled “METHODS AND PRODUCTS FOR ANALYZING NUCLEIC ACIDS USING NICK TRANSLATION”, Ser. No. 60/297,080, the contents of which are incorporated by reference herein in their entirety.

### FIELD OF THE INVENTION

The invention relates to analysis of nucleic acid molecules using nick translation.

### BACKGROUND OF THE INVENTION

DNA strand breaks are involved in various normal cellular processes including replication, DNA repair, and rearrangement (e.g., in rearrangement of immunoglobulin genes). Enzymes capable of inducing as well as correcting (i.e., re-ligating) DNA strand breaks are known. DNA polymerase is an example of an enzyme that corrects breaks, while VDJ recombinase is an example of an enzyme that induces breaks. The action of such enzymes is required for normal cellular and organism development.

DNA strands breaks, however, are also associated with certain types of disorders. The DNA strand break can be the cause of a particular disorder. As an example, it can result from exposure to a known or unknown agent that induces DNA damage. Such damage may be a break in the nucleic acid backbone itself, or it may be a mutation of a nucleotide (e.g., the formation of an adduct) that decreases the stability of the nucleic acid at that region and which ultimately leads to a break in the backbone. In some instances, these breaks can be corrected by the cell. However, if the damage is extensive, it can lead to genomic instability.

The DNA strand break can also be the manifestation of an underlying disorder. As an example, it can result from a mutation in the DNA repair machinery. In a normal cell, the DNA repair pathways seek out and correct genetic lesions such as DNA strand breaks, nicks, and nucleotide modifications. When these DNA repair pathways are impaired, these mutations are not repaired. In some instances, the affected cells cannot undergo division due the sequence specific nicking enzyme to introduce nicks into the nucleic acid molecule, exposing the nucleic acid molecule to a polymerase enzyme and labeled nucleotides, allowing the polymerase enzyme to incorporate labeled nucleotides into the nucleic acid molecule, and detecting a signal from the incorporated labeled nucleotides in the nucleic acid molecule using a linear polymer analysis system.

In one embodiment, the linear polymer analysis system is selected from the group consisting of a Gene Engine™ system, an optical mapping system, and a DNA combing system. In one embodiment, the signal from the incorporated labeled nucleotides is detected using a single molecule detection system. In another embodiment, the nucleic acid molecule is exposed to a station to produce the signal from the incorporated labeled nucleotides.

In this and other aspects of the invention, the nucleic acid molecule may be genomic DNA. In important embodiments, the nucleic acid molecule is a non in vitro amplified nucleic acid molecule. In still other embodiments, the nucleic acid molecule is a single nucleic acid molecule.

In certain embodiments, the sequence specific nicking enzyme can be selected from the group consisting of site-specific nicking enzymes, restriction endonucleases, modified restriction endonucleases, recombination enzymes such as FLP recombinase and Cre recombinase, transposases, engineered protein chimera, DNA repair enzymes including mismatch repair enzymes, helicases, topoisomerases, DNases, modified DNases, homing endonucleases, synthetic restriction enzymes, an viral nickases but are not so limited.

In a related aspect of the invention, the nucleic acid molecule is exposed to a sequence specific endonuclease under conditions that induce single stranded nicks. In other related aspects, the nucleic acid molecule is exposed to a non-sequence specific nicking enzymes under conditions that induce sequence specific nicks.

In other aspects of the invention, the nucleic acid molecule may be exposed to an enzyme that creates nicks on both strands of the double stranded nucleic acid molecule. In this latter aspects, it is preferable that the nucleic acid molecule be exposed to a crosslinking agent. Examples of crosslinking agents include but are not limited to formaldehyde, and UV irradiation, heat, bifunctional crosslinkers, and formamide.

In preferred embodiments, the polymerase is capable of both hydrolyzing a phosphodiester backbone in a 5′ to 3′ direction and synthesizing a phosphodiester linkage in a 3′ to 5′ direction. The polymerase may be DNA polymerase I, or any other polymerase capable of such activity. In other embodiments, the polymerase may be an engineered polymerase that combines a cleavage domain from a DNA degrading enzyme and a polymerase domain from a polymerase. In other embodiments, the polymerase enzyme is selected from the group consisting of DNA polymerase I, Taq polymerase, RNA polymerase, and the like.

In this and other aspects, the labeled nucleotide may comprise a label selected from the group consisting of a fluorescent molecule, a chemiluminescent molecule, a radioisotope, an enzyme substrate, a biotin molecule, an avidin molecule, an electrical charged transducing molecule, a nuclear magnetic resonance molecule, a semiconductor nanocrystal, an electromagnetic molecule, an electrically conducting particle, a ligand, a microbead, a Qdot, a chromogenic substrate, an affinity molecule, a protein, a peptide, an antibody, an antibody fragment, an antigen, a hapten, a nucleic acid, a carbohydrate, and a lipid. In some embodiments, the method further comprises labeling the nucleic acid molecule with a backbone label.

In this and other aspects, the detection system may be selected from the group consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system, and a electromagnetic detection system.

In one aspect, the invention provides a method for identifying a subject having or at risk of developing a disorder are characterized by abnormal nicking of a nucleic acid molecule, comprising determining a nicking pattern of a nucleic acid molecule in a biological sample from a subject, and comparing the nicking pattern of the nucleic acid molecule to a control, wherein a difference in the nicking pattern of the nucleic acid molecule as compared to the control identifies as subject having or at risk of developing the disorder.

In one embodiment, the subject is a human. In another embodiment, the subject has been exposed to a DNA damaging agent. In important embodiments, the nucleic acid molecule is genomic DNA.

In one embodiment, the control is a normal cell. In another embodiment, the control is a set of data from normal cells. In one embodiment, the difference in the nicking pattern is an increase in a total level of nicking. In another embodiment, the difference in the nicking pattern is a decrease in a total level of nicking. In yet another embodiment, the difference in the nicking pattern is a difference in the location of nicking. In one embodiment, the disorder is cancer, such as breast cancer. In another embodiment, the disorder is a DNA repair deficiency disorder, or a disorder associated with susceptibility to DNA damaging agents such as UV irradiation. In preferred embodiments, the nucleic acid is nicked in vivo. In one embodiment, the nucleic acid molecule is further processed by exposing it to a polymerase enzyme and labeled nucleotides, and allowing the polymerase enzyme to incorporate the labeled nucleotides into the nucleic acid molecule.

In yet another aspect, the invention provides a method for screening a compound for the ability to damage a nucleic acid molecule, comprising determining a nicking pattern in a nucleic acid molecule prior to and after exposure of the nucleic acid molecule to a compound, and comparing the nicking pattern prior to and after exposure of the nucleic acid molecule to the compound, wherein the nicking patterns are determined by exposing the nucleic acid molecule to a polymerase enzyme and labeled nucleotides, allowing the polymerase enzyme to incorporate labeled nucleotides into the nucleic acid molecule, and detecting a signal from the incorporated labeled nucleotides in the nucleic acid molecule. In preferred embodiments, the nucleic acid molecule is analyzed and the signal is detected using a linear polymer analysis system.

In one embodiment, the compound is a putative anti-cancer agent. In another embodiment, the method further comprises screening the effect of the compound on normal cells and/or tissues in order to determine its specificity.

In yet a further embodiment, the invention provides a method for assessing the efficacy of a therapeutic treatment, comprising determining a nicking pattern of a nucleic acid molecule from a biological sample from a subject prior to and after the therapeutic treatment, and comparing the nicking pattern prior to the therapeutic treatment with the nicking pattern after the therapeutic treatment, wherein a difference in the nicking pattern as a result of the therapeutic treatment is an indicator of the efficacy of the therapeutic treatment.

In one embodiment, the difference in the nicking pattern is an increase in a total level of nucleic acid nicking. In another embodiment, the difference in the nicking pattern is a decrease in a total level of nucleic acid nicking. In yet another embodiment, the difference in the nicking pattern is a difference in the location of nicking. In another embodiment, the therapeutic treatment is an anti-cancer agent. In a related embodiment, the anti-cancer agent is a DNA damaging agent.

In one embodiment, the nicking pattern is determined by exposing the nucleic acid molecule to a polymerase enzyme and labeled nucleotides, allowing the polymerase enzyme to incorporate labeled nucleotides into the nucleic acid molecule, and detecting a signal from the labeled nucleotides incorporated into the nucleic acid molecule.

In still another aspect, the invention provides a system for optically analyzing a nucleic acid molecule comprising an optical source for emitting optical radiation of a known wavelength; an interaction station for receiving the optical radiation in an optical path and for receiving the nucleic acid molecule that is exposed to the optical radiation to produce detectable signals; dichroic reflectors in the optical path for creating at least two separate wavelength bands of the detectable signals; optical detectors constructed to detect radiation including the signals resulting from interaction of the nucleic acid molecule with the optical radiation; and a processor constructed and arranged to analyze the nucleic acid molecule based on the detected radiation including the signals, wherein the nucleic acid molecule is labeled using nick translation.

In one embodiment, the nucleic acid molecule is a non in vitro amplified nucleic acid molecule. In another embodiment, the nucleic acid is genomic DNA.

In one embodiment, the interaction station includes a slit having a slit width in the range of 1 nm to 500 nm and producing a localized radiation spot. In another embodiment, the slit width is in the range of 10 nm to 100 nm. In one embodiment, the system further comprising a microchannel arranged with the slit to produce the localized radiation spot, the microchannel being constructed to receive and advance the polymer units through the localized radiation spot. In still another embodiment, the system further comprises a polarizer, wherein the optical source includes a laser constructed to emit a beam of radiation and the polarizer is arranged to polarize the beam prior to reaching the slit. In one embodiment, the polarizer is arranged to polarize the beam in parallel to the width of the slit.

In certain embodiments, the nucleic acid molecule is labeled using nick translation that comprises exposing the nucleic acid molecule to a sequence specific nicking enzyme, allowing the sequence specific nicking enzyme to introduce nicks into the nucleic acid molecule, exposing the nucleic acid molecule to a polymerase enzyme and labeled nucleotides, allowing the polymerase enzyme to incorporate labeled nucleotides into the nucleic acid molecule, detecting a signal from the incorporated labeled nucleotides in the nucleic acid molecule using a linear polymer analysis system that comprises a detection system. The nucleic acid molecule may be exposed to the polymerase enzyme and nucleotides, without the need for exposure to the sequence specific nicking enzyme in some embodiments. In these latter embodiments, the nucleic acid molecule is already nicked (e.g., nicked in vivo).

In another aspect, the invention provides a method for analyzing a nucleic acid molecule comprising generating optical radiation of a known wavelength to produce a localized radiation spot; passing a labeled nucleic acid molecule through a microchannel; irradiating the labeled nucleic acid molecule at the localized radiation spot; sequentially detecting radiation resulting from interaction of the labeled nucleic acid with the optical radiation at the localized radiation spot; and analyzing the labeled nucleic acid molecule based on the detected radiation, wherein the nucleic acid molecule is labeled using nick translation.

In one embodiment, the method further comprises employing an electric field to pass the nucleic acid molecule through the microchannel. In another embodiment, the detecting includes collecting the signals over time while the nucleic acid molecule is passing through the microchannel.

In one embodiment, the nucleic acid molecule is labeled using a nick translation approach comprising exposing the nucleic acid molecule to a sequence specific nicking enzyme, allowing the sequence specific nicking enzyme to introduce nicks into the nucleic acid molecule, exposing the nucleic acid molecule to a polymerase enzyme and labeled nucleotides, allowing the polymerase enzyme to incorporate labeled nucleotides into the nucleic acid molecule, detecting the signal from the incorporated labeled nucleotides in the nucleic acid molecule using a linear polymer analysis system that comprises a detection system. In some embodiments, it is not necessary to expose the nucleic acid molecule to the sequence specific nicking enzyme, as the nucleic acid molecule is already nicked (e.g., nicked in vivo).

In yet another aspect, the invention provides a method for analyzing a nucleic acid molecule based on a single stranded nick profile, comprising exposing a nucleic acid molecule to a sequence specific nicking enzyme, allowing the sequence specific nicking enzyme to introduce nicks into the nucleic acid molecule, exposing the nucleic acid molecule to a polymerase enzyme and labeled nucleotides, allowing the polymerase enzyme to incorporate labeled nucleotides into the nucleic acid molecule, and analyzing the nucleic acid molecule for the presence of the label using a linear polymer analysis system, such as but not limited to a Gene Engine™ system, an optical mapping system, and a DNA combing system.

In all of the foregoing aspects, the nucleic acid molecules may be analyzed in either a free form, e.g., in a flow system, or in a fixed form.

All of the foregoing aspects and embodiments of the invention will be explained in greater detail herein.

### DETAILED DESCRIPTION OF THE INVENTION

The invention provides methods, compositions and systems for analyzing nucleic acids based on sequence-specific nick translation to determine patterns of nucleic acid damage and to derive sequence information from such nucleic acids.

Nick translation is a molecular biology technique for incorporating modified nucleotides (e.g., radiolabeled or fluorescently labeled nucleotides) into a nucleic acid molecule (e.g., DNA) (Meinkoth J. and G. M. Wahl, Methods Enzymol 1987; 152:91-94). This is a very efficient method of generating high specific activity radioactive DNA probes with ³²P labeled deoxynucleotides of 10⁸ or greater cpm per g of DNA or high specific activity chemiluminescent or derivatized DNA probes.

In the classical nick translation reaction, the nick is generally made by the action of DNAse. DNAse will attach to DNA at a non-sequence specific site and hydrolyze the phosphodiester bond at that site on one of the DNA strands. In the nick translation reaction, a population of DNA molecules is treated with limiting amounts of DNAse under controlled conditions to nick the DNA. A sufficient density of nicks is created to accommodate the processivity of DNA polymerase I synthetic activity and yet not so high a density of nicks that the DNA will dissociate into oligonucleotides.

In the presence of deoxyribonucleotides, DNA polymerase I will blunt 3′ overhangs and fill in 5′ overhangs but it will have no effect on blunt ends. DNA polymerase I can not “nick-translate” from the ends of double stranded DNA molecules.

In the nick translation reaction, the strand replacement process by DNA polymerase I occurs randomly throughout the DNA molecules due to the essentially random action of DNAse in the nicking process. In order to derive meaningful sequence information from this approach, however, it is desirable that the nicks occur in the nucleic acid molecule in a sequence specific manner.

The invention provides a nick translation based method for sequence specific tagging (i.e., labeling) of nucleic acid molecules (e.g., DNA). The method involves generating sequence specific entry points for a polymerase enzyme (i.e., nicks), along the length of the nucleic acid molecule. This is accomplished by exposing the nucleic acid molecule to a sequence specific nicking enzyme. The method further involves exposing the nicked nucleic acid molecule to a polymerase enzyme capable of incorporating labeled nucleotides such as fluorescently labeled nucleotides or derivatized nucleotides that can be secondarily labeled with, for example, fluorescent or equivalently detectable tags. The method preferably also includes a step of limiting the extent of the labeling or replacement reaction, and if necessary removing the residual nick by ligation or equivalent chemical reaction.

The methods provided herein allow first for the derivation of a nicking pattern for a single nucleic acid molecule. This nicking pattern can be one that is pre-existing in a nucleic acid molecule, for example, as a result of exposure to a DNA damaging agent. The nicking pattern can therefore be used as an indicator of whether a cell or a subject has been exposed to conditions that damage DNA, or whether such a cell or subject is prone to DNA damage. This is the case in subject that carry mutations in DNA repair machinery.

Alternatively, the nicking pattern may be one that is generated in vitro using sequence specific nicking enzymes. In this aspect of the invention, the nicking pattern can be used to derive sequence information about a nucleic acid molecule, given the sequence-specific nature of the nicking enzyme. As described in more detail herein, the position of nicks acts as a marker of the recognition sequence of the nicking enzyme. The position of the nicks are indicated by the position of the incorporated nucleotides. One of the most common forms of nicking enzymes is restriction enzymes which are known to nick nucleic acid molecules such as DNA in a sequence-specific manner. The recognition sequences of a plurality of restriction enzymes are known, and can be obtained by reference to a catalogue of any commercial supplier of molecular biology reagents, such as New England Biolabs.

Once the nicking patterns are determined, they may then be compared to genomic maps for that particular species, to align and orient the nucleic acid molecule and the nicking pattern within the context of the genome. In some embodiments, the superimposition of the nicking pattern with a genomic map also allows for the identification of nicking “hot spots” or mutation “hot spots”. Hot spots are regions of a nucleic acid molecule that contain a higher than average density of nicks, as compared to the nucleic acid molecule as a whole. Nicking hot spots may indicate transcriptionally active regions in the genome. These hot spots can be either already known and identified as genomic loci, or they may be novel. In this latter case, the method leads to the identification of new genes. In both cases, the method provides information about which genetic loci are mutated in vivo, or following exposure to a particular agent (either in vivo or in vitro).

The genomic maps can be obtained for public databases including the Human Genome Project, the results of which are available from the NCBI or NIH websites. These genomic maps can be sequence maps at various levels of resolution, or they can be motif maps, or structural maps, but they are not so limited.

In still other embodiments, as discussed herein, the nicking pattern can be oriented within a single nucleic acid molecule by staining the molecule for known sequences and structures such as telomeres, centromere, repetitive sequences such as Alu repeats, and the like. In still further embodiments, the nucleic acids may be further processed in order to label them for comparison with genomic maps. For example, the nucleic acid maps may be labeled with any label that has been previously used to map that particular genome. The nucleic acid so stained can then be compared to the genomic map generated using the same label. As a specific example, the nucleic acid may be labeled with probes that bind to repetitive sequences, such as Alu repeats, in addition to the nick translation labeling, and then compared with an Alu map of the entire genome in order to determine the location and orientation of the nucleic acid molecule. Once this is determined, the location of the nicks can be determined with respect to the genomic map.

The term “nucleic acid” is used herein to mean multiple nucleotides (i.e., molecules comprising a sugar (e.g., ribose or deoxyribose) linked to an exchangeable organic base, which is either a substituted pyrimidine (e.g., cytosine (C), thymidine (T) or uracil (U)) or a substituted purine (e.g., adenine (A) or guanine (G)). As used herein, the term refers to oligoribonucleotides as well as oligodeoxyribonucleotides. The term shall also include polynucleosides (i.e., a polynucleotide minus a phosphate) and any other organic base containing polymer. Nucleic acid molecules can be obtained from existing nucleic acid sources (e.g., genomic DNA or RNA), or by synthetic means (e.g. produced by nucleic acid synthesis, recombinant DNA techniques, or amplification reactions). The terms “nucleic acid” and “nucleic acid molecule” are used interchangeably.

More specifically, DNA is a double stranded polymer comprising of phosphodiester linked pentose deoxyribose sugars with attached purine or pyrimidine nitrogenous bases. The asymmetry in the pentose sugar leads to a directionality in the phosphodiester linkage whereby the sugar units are linked by phosphodiester bonds between 5′ and 3′ carbons of different sugar units. Thus the two polymer strands of a DNA molecule are anti-parallel. There are two purines, adenine and guanine, and two pyrimidines, thymine and cytosine that constitute the types of nitrogenous bases that are naturally attached to the sugars. Adenine preferentially hydrogen bonds to thymine and cytosine preferentially hydrogen bonds to guanine. The sequence of a nucleic acid molecule refers to the order of the bases along its length. The order of the bases on any one strand determines (or alternatively, is determined by) the order of bases on the other strand by the anti-parallel nature of the DNA strands and by the complementary nature of the hydrogen bonding that can occur between the appropriate purine and pyrimidine bases.

A nucleic acid molecule includes DNA, RNA, and locked nucleic acids and peptide nucleic acids, and is preferably double stranded. DNA includes genomic DNA (such as nuclear DNA and mitochondrial DNA), as well as in some instances cDNA. In important embodiments, the nucleic acid molecule is a genomic nucleic acid molecule. It is to be understood that the reference to DNA in the exemplifications described herein are merely for convenience and clarity, and that any nucleic acid molecule, including those recited above, can be processed and analyzed as described herein. The nucleic acid molecule can be any size, including several nucleotides in length, several hundred, several thousand, and even several million nucleotides in length. In some embodiments, the nucleic acid molecule is the length of a chromosome.

The methods of the invention may be performed in the absence of prior nucleic acid amplification in vitro. In some preferred embodiments, the nucleic acid molecule is directly harvested and isolated from a biological sample (such as a tissue or a cell culture) without the need to amplify the nucleic acid molecule. Accordingly, some embodiments of the invention involve analysis of “non in vitro amplified nucleic acid molecules”. As used herein, a “non in vitro amplified nucleic acid molecule” refers to a nucleic acid molecule that has not been amplified in vitro using techniques such as polymerase chain reaction or recombinant DNA methods.

A non in vitro amplified nucleic acid molecule may, however, be a nucleic acid molecule that is amplified in vivo (e.g., in the biological sample from which it was harvested) as a natural consequence of the development of the cells in the biological sample. This means that the non in vitro nucleic acid molecule may be one which is amplified in vivo as part of gene amplification, which is commonly observed in some cell types as a result of mutation or cancer development. As a result, it is possible to determine the native nicking pattern of a nucleic acid molecule as it existed in vivo. These nicking patterns can yield information regarding the integrity of the genome of the subject from whom the nucleic and molecule was harvested. An above normal level of nicking may indicate that the subject has been exposed to a DNA damaging agent, or alternatively, that the subject has a DNA repair deficiency disorder. A normal level of nicking can be determined by analyzing the level of nicking in either a normal population of subjects, or a normal population of cells.

Harvest and isolation of nucleic acids are routinely performed in the art and suitable methods can be found in standard molecular biology textbooks. The nucleic acid molecule may be harvested from a biological sample such as a tissue or a biological fluid. The term “tissue” as used herein refers to both localized and disseminated cell populations including, but not limited to, brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, sperm, serum, plasma, blood and urine, but are not so limited. Both invasive and non-invasive techniques can be used to obtain such samples and are well documented in the art.

In some embodiments, the invention can be used to analyze nucleic acid derivatives. As used herein, a “nucleic acid derivative” is a non naturally occurring nucleic acid molecule. Nucleic acid derivatives may contain non naturally occurring elements such as non naturally occurring nucleotides and non naturally occurring backbone linkages.

Nucleic acid derivatives may include substituted purines and pyrimidines such as C-5 propyne modified bases (Wagner et al., Nature Biotechnology 14:840- 844, 1996). Purines and pyrimidines include but are not limited to adenine, cytosine, guanine, thymidine, bromo-deoxyuridine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Non-naturally occurring nucleotides, included modified nucleotides such as flourophore-conjugated dNTPs. Some of these non-naturally occurring nucleotides have higher incorporation efficiencies and optionally may be further labeled once incorporated. Other non naturally occurring nucleotides include halogenated nucleotides and amine nucleotides, for instance. Other such modifications are well known to those of skill in the art.

The nucleic acid derivatives may also encompass substitutions or modifications, such as in the bases and/or sugars. For example, they include nucleic acids having backbone sugars which are covalently attached to low molecular weight organic groups other than a hydroxyl group or a thiol (SH) instead of OH at the 3′ position and other than a phosphate group at the 5′ position. Thus, nucleic acid derivatives may include a 2′—O-alkylated ribose group. In addition, nucleic acid derivatives may include sugars such as arabinose instead of ribose. Thus the nucleic acid derivatives may be heterogeneous in backbone composition thereby containing any possible combination of polymer units linked together. In some embodiments, the nucleic acids are homogeneous in backbone composition.

Non naturally occurring backbone linkages include but are not limited to phosphorothioate linkages, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof. The invention also embraces analysis of nucleic acid derivatives that are composed of peptide or locked nucleic acid residues.

The nucleic acid molecules are analyzed using linear polymer analysis systems. A linear polymer analysis system is a system that analyzes polymers in a linear manner (i.e., starting at one location on the polymer and then proceeding linearly in either direction therefrom). As a polymer is analyzed, the detectable labels attached to it are detected in either a sequential or simultaneous manner. When detected simultaneously, the signals usually form an image of the polymer, from which distances between labels can be determined. When detected sequentially, the signals are viewed in histogram (signal intensity vs. time), that can then be translated into a map, with knowledge of the velocity of the nucleic acid molecule. It is to be understood that in some embodiments, the nucleic acid molecule is attached to a solid support, while in others it is free flowing. In either case, the velocity of the nucleic acid molecule as it moves past, for example, an interaction station or a detector, will aid in determining the position of the labels, relative to each other and relative to other detectable markers that may be present on the nucleic acid molecule.

Accordingly, the linear polymer analysis systems are able to deduce not only the total amount of label on a nucleic acid molecule, but perhaps more importantly, the location of such labels. The ability to locate and position the labels (and thus nicks) allows the nicking patterns to be superimposed on other genetic maps, in order to identify the regions of the genome that are affected. In some aspects of the invention, the linear polymer analysis system is a single molecule detection system (i.e., it is capable of analyzing nucleic acid molecules individually).

Other nucleic acid analytical methods which involve elongation of DNA molecule and which have single molecule detection capability can also be used in the methods of the invention. These include optical mapping (Schwartz et al., 1993, Science 262:110-113; Meng et al., 1995, Nature Genet. 9:432; Jing et al., Proc. Natl. Acad. Sci. USA 95:8046-8051) and fiber-fluorescence in situ hybridization (fiber-FISH) (Bensimon et al., Science 265:2096; Michalet et al., 1997, Science 277:1518). In optical mapping, nucleic acid molecules are elongated in a fluid sample and fixed in the elongated conformation in a gel or on a surface. Restriction digestions are then performed on the elongated and fixed nucleic acid molecules. Ordered restriction maps are then generated by determining the size of the restriction fragments. In fiber-FISH, nucleic acid molecules are elongated and fixed on a surface by molecular combing. Hybridization with fluorescently labeled probe sequences allows determination of sequence landmarks on the nucleic acid molecules. Both methods require fixation of elongated molecules so that molecular lengths and/or distances between markers can be measured. Pulse field gel electrophoresis can also be used to analyze the labeled nucleic acid molecules. Pulse field gel electrophoresis is described by Schwartz et al. in Cell, 1984, 37:67. Other nucleic acid analysis systems are described by Otobe et al. (NAR, 2001, 29:109), Bensimon et al. in U.S. Pat. No. 6,248,537, issued Jun. 19, 2001, Herrick and Bensimon (Chromosome Res 1999, 7(6):409-423), Schwartz in U.S. Pat. No. 6,150,089 issued Nov. 21, 2000 and U.S. Pat. No. 6,294,136, issued Sep. 25, 2001.

In some aspects, a Gene Engine™ system is used to interrogate nucleic acid molecules. Gene Engine™ technology is described in greater detail in published PCT patent applications having serial numbers WO98/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other patents, applications and references recited herein are incorporated by reference in their entirety. This system is capable of determining the spatial location of sequence specific incorporation of labeled nucleotides along a nucleic acid molecule. A map of specific sequences within the nucleic acid molecule can be derived from the relative spatial location of the incorporated labeled nucleotides. The spatial location is determined by interrogating linearized nucleic acid molecules, preferably singly, with a detection system that corresponds to the labels on the incorporated nucleotides. The sensitivity of the afore-mentioned system allows single nucleic acids to be studied.

The invention involves the use of sequence-specific nicking enzymes to nick a nucleic enzyme, followed by the use of a polymerase enzyme to fill in such nicks with labeled nucleotides. The location of incorporated labeled nucleotides indicates the location of the sequence recognized by the sequence specific nicking enzyme. The nucleic acid can be nicked with a single nicking enzyme, or it can be nicked with a plurality of nicking enzymes each recognizing a distinct sequence. It is preferable to perform a nicking and polymerase reaction with a pre-determined combination of nicking enzyme and uniquely labeled nucleotide. In this way, the incorporated labels can be distinguished from each other, with each unique label corresponding to a particular sequence. If several nicking enzymes are used, they should be used consecutively, with intervening polymerase reactions.

A “sequence-specific nicking enzyme” is an enzyme that nicks nucleic acids in a sequence-specific manner. “Sequence-specific” as used herein means that the nicking enzyme recognizes a particular linear arrangement of nucleotides or derivatives thereof, and nicks the backbone within that arrangement or in the vicinity of that arrangement. Commonly, the sequence specific nicking enzyme nicks the backbone within the same sequence it recognizes.

Nicking of a nucleic acid molecule means that one of the backbone chains is cleaved, with or without excision of nucleotides. A nick affects only one backbone chain at a given location, although there may be a nick in the other strand of the nucleic acid in the near vicinity. A nucleic acid strand break, on the other hand, refers to cleavage of both backbone chains at the same location. Typical six base recognition restriction enzymes will nick on both strands of the DNA site and depending on the enzyme the nicks will be symmetrically placed about the center of a restriction enzyme recognition site. The result is that a 2, 4 or 6 base overhang exists on dissociation.

At a sufficiently high density of nicks, the double stranded DNA will just dissociate into single strands. However, if the density of nicks is sufficiently low such that the nucleotide distance between nicks is, for example, 4 or more, preferably 10 or more (e.g., 10, 11, 12, 13, 14, 15, 16, 17, 18, 19, 20, 21, 22, 23, 24), or even more preferably 25 or more (e.g., 25, 30, 35, 40, 50) bases, then nicked DNA can retain double stranded integrity.

According to the methods of the invention, nicks are specifically situated or placed such that their location tightly coupled to a sequence motif or site. The nick does not necessarily have to exist within the sequence nor should it be limited to a single nick. Serial nicks on the same strand would effectively be a gap. Gaps may be used as entry points for the polymerase, although, nicks are preferable, in some instances.

The invention envisions several ways for creating sequence specific nicks. These methods are described below. In a first approach, nicking enzymes that are endonucleases can be used. These enzymes recognize specific sequences and cleave the phosphodiester backbone of only one strand of the double helix, at or near such sequences. The cleavage is specific to the recognition site. It is not necessary that the nick be consistently on the same single strand. Rather the nick can sometimes be made on one strand of the double helix and at other times on the other strand of the double helix. The nick needs to located sufficiently close to the sequence so that it does not frustrate the resolution of the detection system. For example, if the detection system has 1 kb resolution then the nick entry point for the polymerase needs to be located such that it does not impact the 1 kb resolution. REBASE (Roberts et al.) enzyme #3759 N.BstNBI is an example of a “nicking” enzyme that recognizes GAGTC and nicks 4 bases 3′ of the recognition site. There are several similar nicking enzymes that can be used in the methods of the invention.

As a second approach, restriction enzymes can be used. Restriction enzymes are endonucleases that recognize specific double stranded DNA sequences and cleave double stranded DNA. Restriction enzymes cleave either within the recognition sequence or at a specific molecular distance away from the recognition sequence (Type II). The cleavage process is effectively a nick on both DNA strands. The nicks can either be directly opposed or displaced to leave a 5′ or 3′ overhang. Generally the displacement is 2, 4 or 6 base pairs. Hydrogen bonding between bases that lie in between nicks that are 2, 4, 6, or less than 20 bases (in some instances) in length may be insufficient to keep the DNA molecule intact. If the nicks are sufficiently displaced (for example, greater than 20 base pairs), then the ends are not free and the integrity of the double stranded molecule is maintained. In instances in which a restriction endonuclease is used which creates nicks on opposite strands that are 2, 4, 6, or less than 20 bases, it may be preferable to crosslink the nucleic acid molecule following the endonuclease reaction, provided that the crosslinking agent does not itself introduce random nicks throughout the length of the nucleic acid molecule. In other embodiments, it may be necessary to stabilize the double-stranded nucleic acid in other ways including reducing temperature, increasing salt concentration, or adding proteins that bind the nucleic acid molecule and thereby stabilize it.

Moreover, in a further embodiment of the invention the restriction enzymes can be used in conditions in which they retain their ability to recognize and bind to sequence specific sites but only nick one of the two DNA strands. Such restriction enzymes (i.e., restriction endonucleases) can be used to create sequence specific nicks without the problem of dissociation of the nucleic acid molecule.

The restriction enzyme can be chemically, enzymatically or genetically modified to lead to endonuclease activity that is predominantly or specifically a “nicking” activity. Reagents, chemicals, proteins, temperature, pressure, metal ions, modifiers etc. can be added to the restriction enzyme reaction to attenuate or modify the cleavage reaction such that the restriction enzyme only nicks one strand rather than created a double stranded break. Alternatively the DNA substrate can be modified directly or by agents such as intercalators or metal ions or reaction conditions such as temperature, ionic strength, pressure etc. to generate only nicks by restriction enzymes. There are ions, chemicals and reactants that will intercalate into the double stranded DNA substrate and change its tertiary structure and hence alter the effectiveness of restriction enzyme cleavage. In yet another embodiment, the DNA template could be modified directly through methylation or other chemistries to limit the extent of cleavage by the restriction enzyme. Agents or conditions can be set up so that the restriction enzyme is limited in its endonuclease activity to nicking just one strand. Examples of modification of the restriction endonuclease reaction conditions have been reported by Taylor J W et al NAR 13:8749-64, 1985; and Kovacs B. J., Gregory S. P. and Butterworth P. H. W. Gene 29:63-68 1984.

A third approach to the generating sequence specific nicking along the length of a nucleic acid molecule involves the engineering restriction enzymes or other DNA modifying enzymes that have sequence specific recognition to nick DNA. The utility of endonucleases for recombinant DNA is due to both their enzymatic activity (generally a double stranded cleavage event) and their sequence specificity. There are naturally occurring endonucleases that have both nicking activity and sequence specificities that are immediately applicable for this invention. However there are also many DNA modifying proteins that can be used if either or both the enzymatic activity or sequence specificity is engineered into them. The enzymatic activity preferably needs to be nicking on a single strand or if on both strands preferably such that there is significant hydrogen bonding to prevent cleavage of the nucleic acid molecule. Sequence specificity needs to be such that the replacement synthesis generated tags are positioned at useful locations (i.e., frequencies) along a DNA polymer. This could mean extending or reducing the length of DNA recognition sites.

An example of an enzyme that has been modified in order to change it from a cleavage enzyme to a nicking enzyme is the EcoRV enzyme. Many Type II restriction enzymes are dimers of two identical subunits that form one DNA binding site and two catalytic units that cleave symmetrically within the recognition sequence. The catalytic site is active only when the restriction enzyme is bound to its double stranded DNA substrate at its specific recognition site. EcoRV has been a prototypical restriction enzyme for study and has been the subject of considerable mechanistic and structural studies and protein engineering. (Stahl F., W. Wende, A. Jeltsch and A. Pingoud PNA 93:6175-6180, 1996.) Stahl et al. made mutations in the catalytic site that reduced endonuclease activity 2-fold, and mutation in the DNA binding domain that decreased the DNA binding activity of the wild type and mutant heterodimer. Stahl et al. found a heterodimer of a catalytic mutant and a DNA binding domain mutant that would bind to EcoRV sites with high affinity and yet just nick one strand of the recognition site. A similar approach can be taken to engineer other restriction endonucleases that recognize different sequences.

Homing endonucleases are enzymes that recognize specific DNA sequence sites of 35 base pairs or more and catalyze a double DNA strand break within the recognition site. These enzymes are involved in insertion and excision of genetic elements, including yeast mitochondrial introns. The family of homing endonucleases can be divided into 4 sub-families characterized by the following motifs: (1) LAGLIDADG, (2) GIY-YIG, (3) H-N-H and (4) His-Cys. Homing endonucleases may be either dimeric or monomeric. One such typical monomeric homing endonuclease, PI-SceI has two copies of LAGLIDADG motif in what appears to be two distinct catalytic subunits. Each subunit was shown to specifically catalyze the cleavage of the top and bottom strand respectively of the double stranded substrate. (Christ F., S. Schoettler, W. Wende, S. Steuer, A. Pingoud and V. Pingoud EMBO J. 1999, 18(24): 69808-6916.) The monomeric homing endonuclease PI-SceI has two catalytic centers for cleavage of the two strands of its DNA substrate. (EMBO 18:6908-6916, 1999.) The two catalytic subunits appear to act independently, suggesting that this enzyme can be engineered into a “nicking” rather than cleaving enzyme.

It is clear that several if not all endonucleases can be engineered in a similar fashion to recognize relevant sites.

A fourth approach to inducing sequence specific nicks in nucleic acid molecules involves synthetic restriction enzymes. DNA binding protein motifs such as zinc finger motifs, homeobox binding domains, lac repressor, GAL, cro etc. can be fused with DNA cleavage domains to construct sequence specific restriction and nicking enzymes. Such chimeric restriction enzymes have been built and described. Yang-Gyun K. and Chandrasegaran S. (PNAS 91:883-887, 1994) reported fusing the Drosophila Ultrabithorax homeodomain to the cleavage domain of Fok I restriction enzyme. More relevantly, chimeric restriction enzymes can be built from fusing zinc finger domains to the cleavage domain of Fok I and thereby building sequence specific restriction enzymes. The cleavage domain of Fok I may require dimerization in order to cleave on both strands of the DNA molecule. It should be possible to modify the cleavage domain such that only one particular phosphodiester bond on one DNA strand is preferentially hydrolyzed or nicked. This may occur when the Fok I cleavage domain is dimerized with a complementary mutated monomer. (Smith et al NAR 28:3361-3369 2000)

Sequence specific DNA binding domains can also be linked to chemical nucleases such as 1,10-phenanthroline-copper. (Pan C. Q., Landgraf R. and Sigman D. S. DNA binding proteins as site specific nucleases Mol Microbiol 1994 12:335-342)

Another approach for introducing sequence specific nicks in a nucleic acid molecule relates to the use of oligonucleotides coupled to reactive groups and metals in order to direct phosphodiester cleavage reactions to sequence specific sites. Dervan et al. reported developing hairpin polyamides composed of N-methylpyrrole (Py) and N-methylimidazole (Im) amino acids that would bind in the minor groove of DNA with sequence specificity. (Mrksich et al 1992 PNAS 89:7586-7590; Wade W. S., Mrksich M. and Dervan P. B. 1992 J. Am. Chem. Soc. 114:8784-8794; Trauger J. W., Baird E. E. and Dervan P. B. 1996 Nature 382:559-561.) Pairing rules determine the side-by-side binding of the aromatic acids. These polyamides can be coupled to protein based or chemical activation and enzymatic domains. Recent work by Mapp A. K., A. Z. Ansari, M. Ptashne and P. B. Dervan (PNAS 2000 97:3930-3935), showed that transcriptional activation domains such as AH tethered to sequence specific polyamide binding agents were biologically effective. Single strand DNA endonucleases or their enzymatic domains could be similarly linked to sequence specific polyamides.

A fifth approach to introducing sequence specific nicks into nucleic acid molecules involves the use of oligonucleotides such as DNAs, RNAs, LNAs or PNAs or chimeras thereof to selectively lock in locally “denatured” double stranded DNA by forming an “R” loop complex and exposing a single select DNA strand to nicking enzymes, endonucleases or chemical nucleases etc. (Chen C. H., Landgraf R., Walts A. D., Chan L., Schlonk P. M., Terwilliger T. C. and Sigman D. S. Chem Biol 1998 5:283-292; Lowell C., Bogenhagen D. and Clayton D. A. Anal Biochem 1978 91:521-531.)

Further methods for introducing sequence specific nicks within a nucleic acid molecule include (1) enzymatic oligonucleotides such as ribozymes that can be designed to recognize sequence specific sites and cleave a single strand of the double stranded DNA; (2) the use of physical conditions such as temperature, pressure, ionic strength and composition, denaturants, organic solvents, inorganic compounds, transition metals etc. to denature DNA and expose sequence selective sites for cleavage, replacement synthesis or labeling; (3) mis-match repair enzymes that will recognize and remove mis-matches; (4) gap repair enzymes; (5) DNA repair enzymes; (6) engineered helicases or topoisomerases. The invention intends to embrace the use of any DNA sequence specific binding and modifying proteins that are capable of nicking phosphodiester bonds at sequence specific locations. Another example includes the exploitation of nucleosome assembly which is sequence specific and sensitive to DNAse treatment e.g. micrococcal nuclease nicking of accessible sites to provide unique entry points for polymerase I. (Inamoto S. et al JBC 266:10086-10092, 1991.)

Recombination can also be used to introduce nicks in a sequence specific fashion. Examples of sequence specific recombination enzymes include FLP (from yeast) or Cre both of which recognize very specific recognition sequences. In the case of Cre the enzyme recognizes a 32 base pair recognition site termed a lox site. Lox sites could be placed at specific locations within a DNA molecule or genome. The Cre enzyme could be engineered so that it recognizes the site and introduces a nick on one strand. The nick could then be an entry site for polymerase I entry.

Transposases can also be used to introduce nicks and subsequently labels at discrete sequence sites. Transposases are enzymes encoded transposons and are involved in enzymatically moving the transposons around in a genome. The sequence specific DNA modifying protein characteristics of the transposons can be exploited to introduce sequence specific nicks for polymerase I directed replacement synthesis reactions.

In yet another embodiment, chromatin can be modified in vivo with chemicals and other modifiers to nick the DNA. In vivo DNA is arranged in structures, broadly described as chromatin, which create an opportunity for structurally discriminating nick introduction. DNA in chromatin is not equally accessible to enzymatic or chemical modification. Thus chromatin structure provides a method of using a nondiscriminatory agent such as DNAses or chemical agents to produce a preferential nicking effect that may have biological relevance. The biologically relevant nicks can be used as polymerase I entry points for labeling and subsequent interrogation of the labels.

In situ nick translation can also be performed in order to generate sequence specific nicks. Genomic DNA in vivo is complexed with chromatin proteins, nuclear proteins, transcription apparatus etc. that lead to differential reactivity to DNAses, restriction enzymes, endonucleases and other DNA modifying enzymes. This differential sensitivity can be used to characterize genomic DNA for linear analysis. Nick translation is used to label the sites for linear DNA analysis. (Tagarro I., Gonzalez-Aguilera J J, Femandez-Peralta A. M., de Stefano G., and Ferrucci L. Genome 1993 36:202-205; de la Torre J., Sumner A. T., Gosalvez J. and Stuppia L. Genome 1992 35:890-894.)

A polymerase enzyme is an enzyme that synthesizes a phosphodiester linkage in a 3′ to 5′ direction. In some instances, the enzyme is also capable of hydrolyzing a phosphodiester backbone in a 5′ to 3′ direction. Examples of polymerase enzymes that can be used in embodiments of the invention include Vent DNA polymerases, Bst DNA polymerase, T7 DNA polymerase, T4 DNA polymerase, DNA polymerase I, DNA polymerase I (Klenow fragment), T7 RNA polymerase, SP6 RNA polymerase, and the like. In preferred embodiments, the polymerase enzyme is DNA polymerase I. In some embodiments, two enzymes can be used, with one having a 3′ to 5′ synthesizing activity and the other having a 5′ to 3′ hydrolyzing activity. In other embodiments, two subunits or domains that do not occur together naturally are engineered or synthesized as domains of a single enzyme.

DNA polymerase I has the ability to degrade one strand of a double stranded DNA polymer in a 5′ to 3′ direction. DNA polymerase I also has the ability to degrade primed single stranded DNA in a 3′ to 5′ direction. This latter ability to degrade a nucleic acid is often referred to as the proofreading property of DNA polymerase I. DNA polymerase I has the property of synthesizing DNA attached to a primer and template. DNA polymerase I synthesizes DNA by catalyzing the linkage of deoxyribonucleotide bases by phosphodiester bonds between the 3′ OH of the primer's 3′ terminal nucleotide to the 5′ phosphate of the incoming deoxyribonucleotide.

DNA polymerase I catalyzes a replacement synthesis reaction on a double stranded DNA template. The template needs to have a nick in the phosphodiester backbone of one of the DNA strands for DNA polymerase I to use as a primer for the synthesis. DNA polymerase I will linearly move along double stranded DNA until it encounters a nick. The exonuclease activity of the DNA polymerase I will sequentially hydrolyze the nicked strand in a 5′ to 3′ direction, effectively removing bases, while linking nucleotides by phosphodiester linkage in a 3′ to 5′ direction. Accordingly, the nick in the DNA strand accompanies the movement of the polymerase along the DNA polymer. If DNA polymerase I is provided with labeled nucleotides, it will incorporate these labeled nucleotides in the process of repairing the link.

Theoretically, DNA polymerase I could continue along the entire length of the nucleic acid molecule, thereby synthesizing long stretches of the nucleic acid molecule. However, it is desirable in some instances to limit the length of nucleotide incorporation by either limiting the nucleotide substrates, diluting the concentration of labeled nucleotide substrates with non-labeled nucleotides, or providing a chain termination compound. For example, a mixture of nucleotides can be used that has a limited amount of one of the four nucleotides. Chain termination would then occur once the limiting nucleotide is depleted. More practically, however, the DNA polymerase I randomly “falls off” (i.e., dissociates from) the nucleic acid molecule. It is also possible to change reaction conditions in order to increase the likelihood of dissociation of DNA polymerase I with the nucleic acid molecule (e.g., introducing a change in the salt concentration).

Other polymerases from prokaryotic and/or eukaryotic organisms with similar endonuclease and polymerase properties to E. coli DNA polymerase I can be used in the methods of the invention. Moreover, although several of the examples provided herein mention DNA polymerase I, it is to be understood that other polymerase enzymes (including combinations of enzymes) that synthesize a phosphodiester linkage in a 5′ to 3′ direction, and optionally hydrolyze a phosphodiester linkage in a 5′ to 3′ direction, are equally suitable.

The nucleic acid molecule of the invention is exposed to the sequence-specific nicking enzyme or the polymerase enzyme when it is brought in contact with these enzymes, to the extent that physical contact can be made and the enzymes can bind to the nucleic acid molecule, and in some instances, scan the molecule for a recognition sequence.

The nucleotides used in the polymerase reaction are preferably labeled with a detectable label. Generally, detection of a label involves absorbance or emission of energy by the label. The label can be detected directly by its ability to emit and/or absorb light of a particular wavelength. An example of direct detection is the use of a fluorophore that absorbs light of a particular wavelength, and emits light of a longer wavelength, Alternatively, the label can be detected indirectly by its ability to bind, recruit and, in some cases, cleave another moiety which itself may emit or absorb light of a particular wavelength. An example of indirect detection is the use of a first enzyme label which cleaves a substrate into visible products.

The label may be of a chemical, peptide, or nucleic acid nature although it is not so limited. Other examples of labels include but are not limited to radioactive isotopes such as P³² or H³, chemiluminescent substrates, chromogenic substrates, luminescent markers such as fluorochromes (e.g., fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), etc.), optical or electron density markers, biotin, avidin, digoxigenin, epitope tags such as the FLAG epitope or the HA epitope, and enzyme tags such as alkaline phosphatase, horseradish peroxidase, β-galactosidase, etc.

Also envisioned by the invention is the use of semiconductor nanocrystals such as quantum dots (i.e., Qdots), described in U.S. Pat. No. 6,207,392 as labels. Qdots are commercially available from Quantum Dot Corporation.

The labels may be directly linked to the nucleotides or may be secondary or tertiary units linked to modified nucleotides. Linkage of labels to nucleotides can be carried out by a number of known covalent and non-covalent processes. These linkages are routine in the art. A universal linkage system that can be used to link a variety of labels to a variety of agents is described by van Gijlswijk et al. (Expert Rev Mol Diagn 2001, 1(1):81-91.)

Analysis of the nucleic acid molecule involves detecting signals from the labels, and determining the position of those signals. In some instances, it may be desirable to further label the nucleic acid molecule with a standard marker that facilitates comparing the information so obtained with that from other nucleic acids analyzed or with genomic maps. For example, the standard marker may be a backbone label, a label that binds to a particular sequence of nucleotides (whether unique or not), or a label that binds to a particular location in the nucleic acid molecule (e.g., an origin of replication, a transcriptional promoter, a centromere, etc.).

One subset of backbone labels are nucleic acid stains that bind nucleic acids in a sequence independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, 85 (orange), SYTO-64, -17, -59, -61, -62, -60, -63 (red).

The linear polymer analysis system is equipped with a detection system that is chosen to correspond to the type of labels used. The labels emit signals that are detected in a spatial or temporal manner. As an example of one suitable system, the Gene Engine™ system allows single nucleic acid molecules to be passed through an interaction station in a linear manner. The nucleotides are interrogated individually in order to determine whether they are conjugated to a detectable label. Interrogation involves exposing the nucleic acid molecule to an energy source such as optical radiation of a set wavelength. In response to the energy source exposure, the detectable label on the nucleotide emits a characteristic detectable signal. The linear polymer analysis system can also be an optical mapping system, such as a DNA combing system.

The mechanism for signal emission will depend on the type of label. The detection system can be selected from the group of detection systems consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system, and a electromagnetic detection system, but is not so limited.

The invention embraces the use of any combination of labels along the length of a nucleic acid molecule. This means that a nucleic acid molecule may be labeled with, for example, a fluorophore, a chromophore, a nuclear magnetic resonance label and a semiconductor nanocrystal along its length and it may be analyzed by the systems described herein. The linear polymer analysis systems have the capability of detecting signals from a number of different “signal modalities”. In one important embodiment, the system uses laser induced fluorescent detection to determine the location of a sequence defined by fluorescent labels.

The sequence-specific information may be either on a single molecule or on a population of molecules. It is not necessary to label all of the sequence specific sites on a molecule. If there is a homogenous population of molecules then it is possible to partially label members of the population and then reassemble the data to generate a complete map for a particular sequence. This method effectively creates a population of single DNA molecule data with a “nested” set of sequence specific data. It does however require knowledge of the distance of the incorporated labeled nucleotides to the recognition sequence of the specific enzyme.

In some embodiments, it may be desirable to crosslink the nucleic acid molecule. Some crosslinking agents can create non sequence specific nicks in a nucleic acid molecule, however. Therefore, it may be preferable to use a crosslinking agent that crosslinks the nucleic acid molecule without introducing any further nicks in the nucleic acid molecule. In other instances, it will be preferable to use a crosslinking agent, after labeling the nucleic acid molecule using the methods of the invention, in order to maintain the double stranded configuration of the nucleic acid molecule, or to crosslink the nucleic acid to a solid support. In this latter instance, the crosslinking agent can itself create nicks in the nucleic acid molecule, however, unless the nucleic acid molecule is then exposed to a polymerase, such nicks will not be labeled.

The following is a brief description of how sequence information can be obtained from a nucleic acid molecule using the methods of the invention. Nucleic acid molecules harvested and isolated from a biological sample (such as a tissue sample or a bodily fluid or an ex vivo tissue culture) are first exposed to an enzyme that is a sequence specific nicking enzyme such as those described herein. The exposure is continued until preferably a majority of the recognition sites recognized by the enzyme are nicked. (In some embodiments, it may not be necessary to perform this first step, however the information derived would generally not be specific to a known sequence, but rather would relate to the nicking pattern already existing in the nucleic acid molecule at the time of harvest.) Following nicking, the nucleic acid molecule is exposed to a polymerase having both synthesis and hydrolysis activity (such as DNA polymerase I) and labeled nucleotides that act as substrates for the polymerase synthesis reaction. Preferably, combinations of sequence specific nicking enzymes and labeled nucleotides are used such that labeling of the nucleic acid molecule with a particular labeled nucleotide indicates the presence of the sequence nicked by the sequence specific nicking enzyme. In some embodiments, and depending upon the frequency of recognition sites for a given enzyme, it may be sufficient to analyze the nucleic acid molecule (for example using the Gene Engine™) after using only one cycle of nicking and labeling. In other embodiments, it may be preferable to perform multiple cycles of nicking and labeling, provided that each cycle uses a particular nicking enzyme and a uniquely labeled nucleotide. The most desirable result is that each incorporated label indicates the position of a particular sequence. In some embodiments, as many nicking enzyme as possible are used in a sequential fashion, with intervening labeling reactions. The result is that the nucleic acid molecule will be labeled with a number of different labels, each corresponding to a particular, known recognition sequence. Both strands of the nucleic acid may be labeled using this technique, and both strands can be analyzed either together or individually.

Each nucleic acid molecule so labeled will have a unique pattern of nicking recognition sites. This unique pattern can be akin to a “fingerprint” of the nucleic acid molecule. The greater the number of different nicking enzymes used (each with a distinct recognition sequence), the more sequence information is available.

The sequencing information derived using the methods of the invention can be compared to genomic sequencing information that is available from sources such as the human genome project. The nicking patterns deduced using the methods of the invention can also be superimposed onto physical genomic maps. These maps (including sequence, motif and structural maps) are available from public sources such as the human genome project, or the genome sequencing projects of other organisms. Superimposition of either or both the sequencing information or the nicking patterns helps to orient such information and thus identify the region of the genome that is being analyzed. The physical maps of genomes are therefore used as references for orienting the nicking patterns determined using the methods of the invention. Moreover, it also helps to identify the genetic loci that are nicked. All aspects of the invention can include the step of comparing the nicking pattern to a physical map of the genome or part thereof for that particular species.

One application of the invention is to determine the propensity of a subject to develop nucleic acid molecule damage such as for example single stranded breaks (i.e., nicks), or alternatively to determine the level of DNA damage that a subject has sustained, and accordingly their ability to repair such damage. In these instance, the methods can be used to identify subjects having or at risk of developing disorders associated with abnormal nicking. Abnormal nicking may be characterized as a level or pattern of nicking that is different from the level or pattern of nicking in a normal nucleic acid molecule from normal cells and/or subjects. Preferably, the normal control is from the same nucleic acid that is being analyzed in the test subject. (For example, chromosome 1 in a test subject is compared to chromosome 1 in the normal control.) In some embodiments, the abnormal nicking is an increase in nicking level over normal. This may be associated with a deficiency in a DNA repair. Examples of DNA repair deficiency disorders such as mismatch repair disorders or chromosomal instability disorders include but are not limited to fragile X syndrome, Fanconi anemia (FA), hereditary non-polyposis colorectal cancer syndrome (HNPCC), ataxia telangiectasia, xeroderma pigmentosa, Nijmegen Breakage syndrome, Cockayne syndrome, trichothiodystrophy, Bloom syndrome, Werner syndrome, and Rothmund-Thomson syndrome. In other embodiments, the abnormal nicking is a decrease in nicking level compared to normal. This may be associated with a deficiency in an enzyme that is normally supposed to nick nucleic acids such as a recombinase involved in immunoglobulin gene rearrangement. In these latter instances, the nucleic acid molecules corresponding to the Ig loci are normally nicked in order to facilitate rearrangement and Ig diversity. An absence or decrease in nicking at these regions can be associated with a lack of recombinases, which can in turn signal an immune system abnormality.

DNA damage can be determined using the nick labeling methods of the invention. In these aspects, it is not necessary that the nicks within the nucleic acid molecules be sequence specific. Rather, the object of the method is to determine the level and potentially the location of such nicks in freshly harvested and isolated nucleic acid molecules. Identifying the location of the nicks will yield information regarding whether DNA damage occurs randomly or non-randomly. Furthermore, regions which are damaged can be isolated and analyzed to characterize any coding sequences contained therein. Agents that are known to introduce nicks into nucleic acids (e.g., DNA) include but are not limited to ionizing radiation, ultraviolet radiation, DNA alkylating agents, hydrogen peroxide, bleomycin, ethyl methane sulfonate, 4-nitroquinoline-N-oxide, etoposide, mitomycin C, reactive oxygen species, and numerous other known DNA damaging agents. The invention intends to embrace analysis of nucleic acids that are nicked, or suspected of being nicked, with known nicking agents, such as those listed herein, as well as putative nicking agents and unknown agents that nick nucleic acids.

The subject may be human or non-human. In preferred embodiments, the subject is human. In other embodiments, particularly those relating to testing of the DNA damaging or DNA repair capacity of agents, the subject is a laboratory animal such as a mouse, rat, rabbit, monkey, fish, and the like. Other subjects suitable to the invention include domestic animals such as dogs, cats, hamsters, etc., agricultural livestock such as horses, cows, pigs, goats, chickens, etc., zoo animals such as zebra, lions, giraffes, bears, etc., and aquaculture species such as finfish and shellfish.

The methods of the invention can be used to determine whether compounds have DNA damaging ability. In one embodiment, the compound being tested is a putative DNA damaging agent, and might be used as a cytotoxic agent in a therapeutic treatment. In another embodiment, the compound being tested preferably does not induce DNA damage, and the method is used as a negative screen in order to eliminate compounds having such an effect. In still other embodiments, the compound is one that may have DNA damage repair activity, and screening methods would focus on decreases in total level of nicking or decreases in nicking in particular regions of the genome.

In experimental systems, it is also possible to correlate DNA damage (as identified using the methods of the invention) with functional assays. As an example, a compound may be introduced into an experimental system (such as a tissue culture, or an animal model), and following exposure to the compound, nucleic acids are harvested and analyzed using the methods of the invention. At the same time functional assays are performed in order to detect any functional defects associated with exposure to the compound. The functional defects so identified can then be correlated to the DNA damage observed in the isolated and analyzed nucleic acid molecules. More specifically, the damaged regions of the nucleic acid molecule might be involved in the particular function being analyzed, and further analysis of that particular region would then be warranted.

Another application of the methods described herein is the assessment of the efficacy of therapeutic treatments. In an important embodiment, the therapeutic treatment is the administration of an anti-cancer agent. In other embodiments, the therapeutic treatment is a DNA damaging agent. DNA damaging anti-cancer agents include agents such as topoisomerase inhibitors (e.g., etoposide, ramptothecin, topotecan, teniposide, mitoxantrone), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), and radiation therapy. In other embodiments, the therapeutic treatment is intended to compensate for a DNA repair deficiency, and thus its efficacy can be indicated by a decrease in the total level of nicking in the genome as a whole or in select regions of the genome.

The invention provides a method whereby a sample can be harvested from a subject either diagnosed with a particular disorder (such as for example cancer) or a subject at risk of developing such a disorder. The sample may be a tissue, a cell population or a bodily fluid, and would usually be acquired by a biopsy from the subject. Nucleic acid molecules from the sample are harvested and isolated and analyzed to determine their nicking patterns, according to the methods of the invention. A “pre-treatment” nicking pattern of one, more than one or all nucleic acid molecules can be so determined. The subject would then be treated with the therapeutic treatment and following such treatment, another biological sample would be harvested from the subject. Nucleic acid molecules are harvested and isolated from the “post-treatment” sample, and analyzed to determine their nicking pattern. Preferably, the samples are harvested from the same tissue, region of the body, or bodily fluid. For example, if the subject has a tumor, both the pre-treatment and post-treatment samples would derive from the tumor. Generally, the samples will be taken from those cells, tissues, or fluids thought to be affected by the disorder. In other instances, however, it may be desirable to investigate the effect of the therapeutic treatment on non-diseased cells or tissues. For example, it may be desirable to determine the specificity of particular therapeutic treatments in order to identify treatments that more specifically target diseased cells or tissues while leaving normal cells or tissues intact.

In some of the above-noted aspects, it is preferable to compare nicking patterns with control nicking patterns, generally in the form of normal cells, normal tissues, normal subjects, or data generated from any of the above. The normal level can also be a range, for example, where a population is used to obtain a baseline range for a particular group into which the subject falls. The normal value can depend upon a particular population selected. Preferably, the normal levels are those of apparently healthy subjects who have no prior history of nicking-mediated disorders. More preferably, the normal level is that level in a tissue of a normal subject corresponding to the tissue sampled for the test subject. As an example, melanoma spots are, in some cases, sufficiently delineated to the extent that they can be distinguished from surrounding normal skin. This delineation facilitates selective removal of diseased tissue, and can be used in the present invention to harvest both suspected diseased tissue and normal tissue from a given subject. Such normal levels or normal patterns, then can be established as preselected values or patterns, taking into account the category in which an individual falls. Appropriate ranges and categories can be selected with no more than routine experimentation by those of ordinary skill in the art. Either the mean or another preselected number within the range can be established as the normal preselected value.

Importantly, nicking patterns can also be compared in terms of the position of the nicks in the nucleic acid molecules. Differences between nicking patterns of nucleic acid molecules from subjects or cells known to be diseased and control or normal subjects or cells can lead to the identification of loci that are mutated in particular disorders.

### References

Soslau G. and Pirollo P. 1983 Selective inhibition of restriction endonuclease cleavage by DNA intercalators Biochem Biophys Res Commun 1983 115(2): 484-491

Osterlund M, Luthman H. Nilsson S V, Magnusson G. Ethidium-bromide inhibited restriction endonucleases cleave one strand of circular DNA Gene 1982 20(1):121-125

Nakatani K., Sando S., Saito I. Photoinduced cleavage of single and double stranded DNA at single guanine proximal to target sequence by dibenzoyldiazomethane-ODN conjugate Nucleic Acids Symp Ser 37, 85-6, 1997

Pan C Q, Landgraf R., Sigman D S DNA-binding proteins as site specific nucleases Mol Microbiol 1994 12(3):335-42

Kittler L., Bell A., Baguley B C., Lober G. Sequence specific modulation of DNA restriction enzyme cleavage by minor groove binders Biol Chem 379: 4-5 1998

Fukuda H., Ohtsuba E, Roles of TraI protein with activities of cleaving and rejoining the single-stranded DNA in both initiation and termination of conjugal DNA transfer Genes Cells 2:735-751 1997

Cost G J. Golding A, Schlissel M S, Boeke J D Target DNA chromatinization modulates nicking by L1 endonuclease NAR 29:573-577 2001 L1 endonuclease (L1 EN) nicks genomic DNA as step in transposition reaction

Champoux J J DNA topisomerase I mediated nicking of circular duplex DNA Methods Mol Biol 2001 95:81-87

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

I claim:

1. A method for analyzing a nucleic acid molecule, comprising:
exposing a nucleic acid molecule to a sequence specific nicking enzyme,
allowing the sequence specific nicking enzyme to introduce nicks into the nucleic acid molecule,
exposing the nucleic acid molecule to a polymerase enzyme and labeled nucleotides,
allowing the polymerase enzyme to incorporate labeled nucleotides into the nucleic acid molecule, and
detecting a signal from the labeled nucleotides incorporated into the nucleic acid molecule.

2. The method of claim 1, wherein the signal is detected using a linear polymer analysis system.

3. The method of claim 2, wherein the linear polymer analysis system is a single molecule detection system.

4. The method of claim 2, wherein the linear polymer analysis system is selected from the group consisting of a Gene Engine™ system, an optical mapping system, and a DNA combing system.

5. The method of claim 1, wherein the nucleic acid molecule is genomic DNA.

6. The method of claim 1, wherein the nucleic acid is a non in vitro amplified nucleic acid molecule.

7. The method of claim 1, wherein the nucleic acid molecule is a single nucleic acid molecule.

8. The method of claim 1, wherein the nucleic acid molecule is exposed to a station to produce the signal from the labeled nucleotides incorporated into the nucleic acid molecule.

9. The method of claim 1, wherein the labeled nucleotide comprises a label selected from the group consisting of a fluorescent molecule, a chemiluminescent molecule, a radioisotope, an enzyme substrate, a biotin molecule, an avidin molecule, an electrical charged transducing molecule, a nuclear magnetic resonance molecule, a semiconductor nanocrystal, an electromagnetic molecule, an electrically conducting particle, a ligand, a microbead, a chromogenic substrate, an affinity molecule, a Qdot, a protein, a peptide, a nucleic acid, a carbohydrate, an antibody, an antibody fragment, an antigen, a hapten, and a lipid.

10. The method of claim 1, wherein the detection system is selected from the group consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system, and a electromagnetic detection system.

11. The method of claim 1, further comprising labeling the nucleic acid molecule with a backbone label.

12. The method of claim 1, wherein the polymerase enzyme is DNA polymerase I.

13. The method of claim 1, wherein the sequence specific nicking enzyme is selected from the group consisting of restriction endonucleases, modified restriction endonucleases, recombination enzymes, recombinase, transposases, engineered protein chimera, DNA repair enzymes including mismatch repair enzymes, helicases, topoisomerases, DNases, modified DNases, homing endonucleases, and synthetic restriction enzymes.

14. A method for analyzing a nucleic acid molecule, comprising:
determining a nicking pattern of a nucleic acid molecule in a biological sample from a subject, and
comparing the nicking pattern of the nucleic acid molecule to a control.

15. The method of claim 14, further comprising determining a difference in the nicking pattern of the nucleic acid molecule as compared to a control.

16. The method of claim 15, wherein a difference in the nicking pattern of the nucleic acid molecule as compared to the control identifies a subject having or at risk of developing a disorder characterized by abnormal nicking of a nucleic acid molecule.

17. The method of claim 16, wherein the subject is a human.

18. The method of claim 14, wherein the nucleic acid molecule is genomic DNA.

19. The method of claim 16, wherein the subject has been exposed to a DNA damaging agent.

20. The method of claim 14, wherein the control is a normal cell.

21. The method of claim 14, wherein the control is a set of data from normal cells.

22. The method of claim 16, wherein the difference in the nicking pattern is an increase in a total level of nicking.

23. The method of claim 16, wherein the difference in the nicking pattern is a decrease in a total level of nicking.

24. The method of claim 16, wherein the difference in the nicking pattern is a difference in the location of nicking.

25. The method of claim 16, wherein the disorder is cancer.

26. The method of claim 25, wherein the cancer is breast cancer.

27. The method of claim 16, wherein the disorder is a DNA repair deficiency disorder.

28. The method of claim 14, wherein the nucleic acid molecule is a non in vitro amplified nucleic acid molecule.

29. The method of claim 14, wherein the nucleic acid molecule is nicked in vivo.

30. The method of claim 29, wherein the nicking pattern is determined by
exposing the nucleic acid molecule to a polymerase enzyme and labeled nucleotides,
allowing the polymerase enzyme to incorporate labeled nucleotides into the nucleic acid molecule, and
detecting a signal from the labeled nucleotides incorporated into the nucleic acid molecule.

31. The method of claim 30, wherein the polymerase enzyme is DNA polymerase I.

32. A method for screening a compound for the ability to damage a nucleic acid molecule, comprising
determining a nicking pattern in a nucleic acid molecule prior to and after exposure of the nucleic acid molecule to a compound, and
comparing the nicking pattern prior to and after exposure of the nucleic acid molecule to the compound,
wherein the nicking patterns are determined by
exposing the nucleic acid molecule to a polymerase enzyme and labeled nucleotides,
allowing the polymerase enzyme to incorporate labeled nucleotides into the nucleic acid molecule, and
detecting a signal from the labeled nucleotides incorporated into the nucleic acid molecule.

33. A method for assessing the efficacy of a therapeutic treatment, comprising:
determining a nicking pattern of nucleic acid molecule from a biological sample from a subject prior to and after the therapeutic treatment, and
comparing the nicking pattern prior to the therapeutic treatment with the nicking pattern after the therapeutic treatment,
wherein a difference in the nicking pattern as a result of the therapeutic treatment is an indicator of the efficacy of the therapeutic treatment.

34. The method of claim 33, wherein the difference in the nicking pattern is an increase in a total level of nucleic acid nicking.

35. The method of claim 33, wherein the difference in the nicking pattern is a decrease in a total level of nucleic acid nicking.

36. The method of claim 33, wherein the therapeutic treatment is an anti-cancer agent.

37. The method of claim 36, wherein the anti-cancer agent is a DNA damaging agent.

38. The method of claim 33, wherein the nicking pattern is determined by
exposing the nucleic acid molecule to a polymerase enzyme and labeled nucleotides,
allowing the polymerase enzyme to incorporate labeled nucleotides into the nucleic acid molecule, and
detecting a signal from the labeled nucleotides incorporated into the nucleic acid molecule.

39. A system for optically analyzing a nucleic acid molecule comprising:
an optical source for emitting optical radiation of a known wavelength;
an interaction station for receiving the optical radiation in an optical path and for receiving the nucleic acid molecule that is exposed to the optical radiation to produce detectable signals;
dichroic reflectors in the optical path for creating at least two separate wavelength bands of the detectable signals;
optical detectors constructed to detect radiation including the signals resulting from interaction of the nucleic acid molecule with the optical radiation; and
a processor constructed and arranged to analyze the nucleic acid molecule based on the detected radiation including the signals,
wherein the nucleic acid molecule is labeled using nick translation.

40. The system of claim 39, wherein the nucleic acid molecule is labeled with a label selected from the group consisting of a fluorescent molecule, a chemiluminescent molecule, a radioisotope, an enzyme substrate, a biotin molecule, an avidin molecule, an electrical charge transducing molecule, a nuclear magnetic resonance molecule, a semiconductor nanocrystal, an electromagnetic molecule, an electrically conducting microparticle, a protein, a peptide, an antibody, an antigen, an antibody fragment, a hapten, a ligand, a Qdot and a microbead.

41. The method of claim 39, wherein the nucleic acid molecule is a non in vitro amplified nucleic acid molecule.

42. The method of claim 39, wherein the nucleic acid molecule is genomic DNA.

43. The system of claim 39, wherein the interaction station includes a slit having a slit width in the range of 1 nm to 500 nm and producing a localized radiation spot.

44. The system of claim 43, wherein the slit width is in the range of 10 nm to 100 nm.

45. The system of claim 43, wherein further comprising a microchannel arranged with the slit to produce the localized radiation spot, the microchannel being constructed to receive and advance the polymer units through the localized radiation spot.

46. The system of claim 45, further comprising a polarizer, wherein the optical source includes a laser constructed to emit a beam of radiation and the polarizer is arranged to polarize the beam prior to reaching the slit.

47. The system of claim 46, wherein the polarizer is arranged to polarize the beam in parallel to the width of the slit.

48. The method of claim 39, wherein the nucleic acid molecule is labeled using nick translation comprising
exposing the nucleic acid molecule to a polymerase enzyme and labeled nucleotides, and
allowing the polymerase enzyme to incorporate labeled nucleotides into the nucleic acid molecule.

49. The method of claim 48, further comprising
exposing the nucleic acid molecule to a sequence specific nicking enzyme,
allowing the sequence specific nicking enzyme to introduce nicks into the nucleic acid molecule, prior to exposing the nucleic acid molecule to the polymerase enzyme.

50. The method of claim 48, wherein the labeled nucleotides are detected using a detection system selected from the group consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system, and a electromagnetic detection system.

51. A method for analyzing a nucleic acid molecule comprising:
generating optical radiation of a known wavelength to produce a localized radiation spot;
passing a labeled nucleic acid molecule through a microchannel;
irradiating the labeled nucleic acid molecule at the localized radiation spot;
sequentially detecting radiation resulting from interaction of the labeled nucleic acid with the optical radiation at the localized radiation spot; and
analyzing the labeled nucleic acid molecule based on the detected radiation,
wherein the nucleic acid molecule is labeled using nick translation.

52. The method of claim 51, further comprising employing an electric field to pass the nucleic acid molecule through the microchannel.

53. The method of claim 51, wherein the detecting includes collecting the signals over time while the nucleic acid molecule is passing through the microchannel.

54. The method of claim 50, wherein the nucleic acid molecule is labeled using a nick translation approach comprising
exposing the nucleic acid molecule to a polymerase enzyme and labeled nucleotides, and
allowing the polymerase enzyme to incorporate labeled nucleotides into the nucleic acid molecule.

55. The method of claim 54, further comprising
exposing the nucleic acid molecule to a sequence specific nicking enzyme,
allowing the sequence specific nicking enzyme to introduce nicks into the nucleic acid molecule, prior to exposing the nucleic acid molecule to the polymerase enzyme.

56. The method of claim 54, wherein the labeled nucleotides are detected using a detection system selected from the group consisting of a fluorescent detection system, an electrical detection system, a photographic film detection system, a chemiluminescent detection system, an enzyme detection system, an atom force microscopy (AFM) detection system, a scanning tunneling microscopy (STM) detection system, an optical detection system, a nuclear magnetic resonance (NMR) detection system, a near field detection system, a total internal reflection (TIR) system, and a electromagnetic detection system.

57. The method of claim 54, wherein the labeled nucleotides are conjugated to a label selected from the group consisting of a fluorescent molecule, a chemiluminescent molecule, a radioisotope, an enzyme substrate, a biotin molecule, an avidin molecule, a Qdot, an electrical charge transducing molecule, a nuclear magnetic resonance molecule, a semiconductor nanocrystal, an electromagnetic molecule, a protein, a peptide, an antibody, an antibody fragment, an antigen, a hapten, a ligand, and a microbead.

58. The method of claim 51, wherein the nucleic acid molecule is a non in vitro amplified nucleic acid molecule.

59. The method of claim 51, wherein the nucleic acid molecule is genomic DNA.
